(19) United States
(12) Patent Application Publication
(10) Pub. No.: US 2011/0177174 A1
(43) Pub. Date: Jul. 21, 2011

(54) Fruit chocolate

(76) Inventors: Brian Crowley, Jersey City, NJ (US); Christopher Najmeddine, Jersey City, NJ (US)

(21) Appl. No.: 12/930,796

(22) Filed: Jan. 18, 2011

Related U.S. Application Data
(60) Provisional application No. 61/295,860, filed on Jan. 18, 2010.

(51) Int. Cl. A61K 36/185; A23G 1/00; A23G 1/42; A23G 1/48; A23G 1/54; A61P 3/02

(52) U.S. Cl. 424/725; 426/631; 426/61; 426/72; 426/93

(57) ABSTRACT

An intimately incorporated meltaway confection highlighting a fruit/fruit blend whose primary fat constituent is cocoa butter, having anhydrous, powdered freeze-dried fruit/fruit juices, and other fruit essences and oils, along with dairy and nutritive carbohydrate sweeteners. Additional flavorings, emulsifiers and fruit based acids may be added to further enhance palatability. Fruit/botanical blends would be achieved by adding an anhydrous powdered botanical component prior to conching. The conching process would employ a constant conching pressure for all aforementioned components of the present invention in order to create a smooth texture and mouth-feel. Further optional components comprise inclusions and coatings typical of other chocolate confections, in addition to metabolic compounds and medicinals for further health benefits.

### CROSS-REFERENCE TO RELATED APPLICATIONS

This application claims the benefit of provisional patent application Ser. No. 61/295,860, filed 2010 Jan. 18 by the present inventors.

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to chocolate products, based on cocoa butter as the sole principal fat constituent, specifically with powdered freeze-dried fruit components intimately included therein. Additionally the products of the present invention have nutritive carbohydrate sweeteners and dairy components included therein. The resulting chocolate product may have similar attributes as those of traditional chocolate, except that the anhydrous, freeze-dried fruit powders comprise the solids mass typically comprised of cocoa solids.

2. Prior Art

The use of chocolate and fruit in combination in the confectionery industry is well documented. Indeed fruit oils and fat-soluble flavorings have been used to flavor chocolate, thus offering the consumer a fruit flavor impression that complements the chocolate.

U.S. Pat. No. 6,641,857^{[a]} issued in November of 2003 claims an edible anhydrous chocolate-based fat system, that may be light chocolate, dark chocolate, or white chocolate, and includes an additional sweet flavor which is arrived at by infusing or saturating the sugar component of the fat system with an essential oil of the chosen flavor.

Even real fruit, be it fresh or dried through various means have been featured in chocolate confections, typically in pieces that retain a distinction from the chocolate itself and act as a complementary component. Because of the moisture content of the fruit it was not possible to truly incorporate it into a cocoa butter based confection.

U.S. Pat. Application #20080299275^{[b]} claims the inclusion of freeze-dried fruit into a vegetable fat based confectionery coating or chip. However, only 0.5%-3.0% by weight of the freeze dried fruit component is used—a stipulation in its first claim, and a range that the current inventors regard as an extremely limiting percentage. Also in the wording of its first claim, the product is solely “for use as a confectionery coating or chip in food products”, and is as such inherently claimed as an additive component to another food product.

Additionally, only certain types of fruit are claimed, including raspberries, strawberries, blueberries, blackberries, loganberries, lingonberries, cranberries, red currants, black currants, mangoes, pineapples, peaches, apricots, and nectarines; other fruits are either ignored or outright rejected, such as melons and starfruit.

As well, U.S. Pat. Application #20080299275 describes, by way of preparation of the product, that the freeze dried fruit particles are either simply admixed, or admixed and further conched, but at a lower pressure than the vegetable fats were conched, with particles reaching up to 3 mm in width. This however, results in a textural mouth-feel. The anhydrous fruit granule would ‘infuse’ the corresponding vegetable fat with its flavor and flavonoids—as a brewed cup of tea, with the tea leaves retaining their distinction from the cup of brew as an entirety.

Further, it fails to claim the addition of flavoring agents to enhance the fruit flavor. In fact, in Claim #8 where cocoa is not present, and therefore most akin to the current invention, vanilla is the only claimed flavoring agent—as is in most commercially produced white chocolates—and thus gains its fruit flavor solely from the freeze-dried components. [Notable is that vanilla is regarded as an optional ingredient according to the U.S. Food and Drug Administration (FDA).]

It is the belief of the current inventors that these points pose a major olfactory shortcoming to the consumer. In practice, the fruit particles merely become coated by the vegetable fat. The resulting product does not become intimately incorporated, and the fruit component features in a complementary role to the vegetable fat base. Because the flavor molecules of the fruits are typically water soluble, their ability to ‘infuse’ the corresponding vegetable fat constituent is hindered greatly through a manufacturing process that employs lower conching pressures or simple admixture incorporations of such low volumes of fruit particles.

U.S. Pat. No. 4,792,449^{[C]} issued in December 1988 claims a method for administering drugs, involving solubilizing a drug in acceptable foods admixed with an oil. U.S. Pat. No. 7,048,941^{[d]} issued in May of 2006 claims a chocolate composition that acts as a carrier for nutrient- and/or medicament-containing compositions designed to treat various ailments/health conditions through ingestion.

### BACKGROUND OF THE INVENTION

### Objects and Advantages

In March 2007, The Grocery Manufacturers Association lobbied the federal government to permit confections containing cheaper hydrogenated vegetable oil in place of cocoa butter to be sold as “chocolate”.^{[e,f]} In June 2007, as a response to consumer concern after the proposed change, the FDA re-iterated that “Cacao fat, as one of the signature characteristics of the product, will remain a principal component of standardized chocolate”^{[g]}

On Oct. 4, 2002 the FDA established a standard of identity for white chocolate, and in essence made cocoa butter the only common ingredient in, and that which defines all chocolates. Further, it makes no mention of an inclusion of fruit as an optional ingredient, and thus the current product is not a white chocolate. As such, the novelty in the description of the current product may be sufficient to warrant the establishment of a new standard in the U.S. Code of Federal Regulations, Title 21, Chapter 1, Part 163—Cocoa Products—Subpart B.^{[h]}

The current inventors have made efforts to create an intimately incorporated fruit imbued cocoa butter based meltaway confection that highlights a fruit, fruit blend, or fruit botanical blend, creating a product that is by definition a ‘chocolate’, thus a fruit chocolate.

The first advantage over prior art is in obtaining the fruit component. The process in which the fruit is prepared as per U.S. Pat. Application #20080299275 is that whole fruits are at first freeze-dried and then unwanted elements, such as seeds, are sifted out. This process, while understandably cuts down on the possibility of oxidation of the fruits' flesh and nutrients, would not be appropriate to describe the preparation regarded as optimal for all culinary fruits—in fact some fruit components of the current invention would be best prepared by juicing or otherwise omitting undesirable elements (seeds, seed casings, excess fiber, etc.) of the fruit prior to freeze drying in an oxidation free environment. Some of these juices may further require anti-caking agents, such as maltodextrin—a saccharide that is added prior to the freeze drying phase-in order to produce an optimal anhydrous fruit constituent for milling into a superfine powder and incorporating into the fat base.

The fundamental difference, however, is in the homogenization of the product. The more highly concentrated freeze-dried powdered fruit particles of the current invention are measured in increments over 1000 times smaller, and in percentages that may be over 13 times more than those of U.S. Pat. Application #20080299275. Characteristically, the present invention's blend of fat and solids might be well compared to a potter's slip, wherein the particles are intimately incorporated into the principal fat resulting in a consistency somewhere between a paste and a suspension.

Further, the addition of fruit based acids, considered by the current inventors necessary to achieve a ‘fresh’ fruit flavor in most applications, may be one of the most poignant differences between the present invention and that of U.S. Pat. Application #20080299275. The fruit, supplemented by the intimate incorporation of fruit acids, lends the product to a pH typically less than 7.0. Due to the nature of the current product, a salivation response is triggered by both the sugar and the acids when consumed, and as such provides for an experience and flavor hitherto unknown in chocolate products.

In the quest to create a smooth ‘chocolate’ confection that highlights a fruit, its has also been observed by the current inventors that the addition of fat soluble fruit flavorings matching the fruit component significantly improves an olfactory response in the consumer, while also significantly enhancing the taste.

In addition to highlighting a fruit or fruit blend, other culinary ingredients from the natural world are claimed that could lead to a whole host of flavor combinations provided their inclusion be in an anhydrous state prior to intimate incorporation as, by nature, cocoa butter is hydrophobic.

Still further objects and advantages will become apparent from a consideration of the ensuing description.

### SUMMARY

Featured prominently in the many variants of the current invention are powdered freeze-dried fruit/fruit juice, cocoa butter, milk and sugar. The combination of and the process by which such ingredients are prepared and combined are that which makes this product unique. The novelty of the process and method described herein is in reducing the fruit component into a particle size smaller than the tongue can detect, while producing a healthful cocoa butter-based confection.

The mechanical, or combination human/mechanical, processing of the fruit imparts efforts to include the correct balance of flesh and juice, with the exclusion of undesirable fruit elements such as seeds, seed casings, stems and/or excess fiber. It is then freeze-dried, and finally pulverized and milled into a superfine powder of particle size <50 μm. Some fruits that have been at first juiced may be processed with the inclusion of additional saccharides, such as malto-dextrin, added as anti-caking agents prior to the freeze-drying phase. This pure freeze-dried fruit powder would take the place of the cocoa solids (defatted or “Dutched” (alkali processed) cocoa powder, or chocolate liquor) as is used in the processing of traditional chocolate candy.

The conching process is employed to further reduce the particle size of all components of the current invention creating a homogeneity and smooth mouth-feel.

Additionally anhydrous powdered botanical constituents may be added for even further flavor profiles. The range of flavors, therefore, is limited only by the total number of pure fruits, fruit blends and fruit/botanical blends available.

The healthful benefits are also variable depending on the intimately incorporated components.

### DRAWINGS

FIG. 1—Flowchart

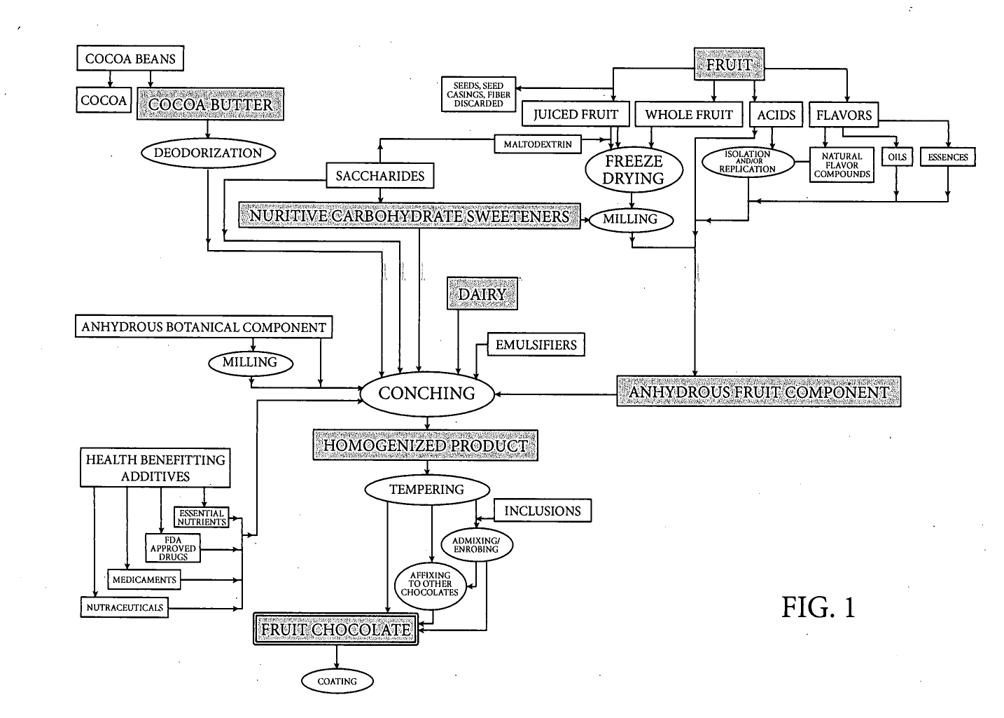

### DETAILED DESCRIPTION

The present inventors have discovered that if powdered freeze-dried fruit/fruit juices are intimately included in the cocoa butter base of a chocolate candy, similar to white chocolate, and in lieu of cocoa solids, then the flavor of the fruit which has been incorporated into the cocoa butter fat base will feature most prominently in the product. As the intent is to highlight a fruit in the present invention, so the cocoa butter shall be deodorized, preferably in all applications of such.

The fat base of the present invention emulates the principal fat constituent of other types of ‘chocolate’. It follows that the preparation and manufacture of the present cocoa butter based product, in keeping with the present invention, will utilize conventional chocolate making processes and apparatus. That is to say the usual chocolate making steps, particularly after the separation of cocoa butter from the cocoa liquor, including the conching process are employed.

Particularly, the manufacturing process will emulate the manufacturing process of other various kinds of chocolate, by varying the amount of sugar and milk products, in conjunction with the powdered freeze-dried fruit components and other ingredients that may be employed in its manufacture.

It, however, must be stressed that the fruit constituent must have at first been freeze-dried. Advantageous of freeze-dried fruit is that it is anhydrous, and typically can be rendered to have a superfine powder-like consistency. Its anhydrous state allows for the incorporation into a lipid base, which is by nature hydrophobic. It has been found that the addition of certain saccharides such as maltodextrin, in varying proportions aids in the preparation of the fruit constituent by acting as an anti-caking agent prior to the freeze drying process, in particular when the fruit has been juiced.

Characteristic of freeze-dried fruit/fruit juice is that there is typically a high concentration, in both flavor as well as health benefits, of the fruit present in the freeze-dried product. As such the cocoa butter based confections may be regarded as a unique vehicle by which to enjoy both the true flavor and benefits of any of the various culinary fruits, fruit blends or fruit/botanical blends. For example, a pomegranate or acai fruit chocolate may be heralded as antioxidant rich, and a watermelon fruit chocolate as good source of lycopene.

A brief description of the process of freeze-drying fruit now follows.

It should first be noted that freeze-dried fruit/fruit juice is fruit/fruit juice from which the water content has been sublimated. The freezing step is one in which the fruit component is taken to a temperature and pressure lower than the triple point at which the solid and liquid phases of the fruit can exist. By increasing the temperature above 0° C., while keeping the pressure below 0.06 atmospheres (ATM), the water inside the fruit component sublimates directly from a solid to a gas, bypassing the liquid phase, and thus drying the fruit leaving behind the water soluble sugars, fibers, and flavor constituents.

The freeze-drying of fruit is carried out under vacuum. A primary drying phase may remove as much is 98% of water in the fruit by sublimation; an additional drying phase is utilized to sublimate the remaining approximately 2% of water. Typically, once the fruit has been freeze-dried, the vacuum is broken with an inert gas such as nitrogen, and the freeze-dried fruit product is further treated for purposes of the present invention.

Furthering the treatment of the freeze-dried fruit, it is mechanically processed so as to reduce the freeze-dried fruit to a powder whose particle size is typically <50 μm; it may be processed with the inclusion of sugar crystals sufficient enough to act as both a desiccant as well as a milling aide.

The fruit powder is then added to the cocoa butter base. The percentage of the product which is comprised of the powdered freeze dried fruit/fruit juice is dependent on each fruit's individual flavor intensity, fiber content and sugar content, and is <40%.

Along with the milk products, any additional sugars, emulsifiers, fruit based acids, essences, oils and flavoring agents necessary for optimum palatability are added to the cocoa butter base during the conching process.

In the case the product has further included a botanical element (other than the developed ovary of a seed plant), or an additional saccharide, the inclusion of such additional components are of a percentage consistent with producing a product of superior palatability. Such additions may be included for a variety of reasons, including flavor, elevated sweetness levels, reduced calorie variants (stevia extracts), [etc.].

Safe and natural coloring agents may also be added to aid in the differentiation of the chocolate product due to the neutrally/similarly shaded flesh of many fruits.

Botanical components utilized to increase the health benefits of the current invention to the consumer, such as neutraceuticals, and/or compounds or other pharmaceuticals created in a lab may also be added. The product labeling must clearly denote such additive with an appropriate statement, ie. “With_added”, or “_added”.

Further processing, via the conching process, is employed to not only assure complete coating of every solid particle with cocoa butter, but to also further reduce the solids particle size, measured in terms of microns, to preferably <15 μm, with an ideal size <2 μm. The intense conching process renders the fruit particles smaller than the tongue can detect, resulting in a state of homogeneity. The longer the conching process continues the smoother the end product becomes.

Tempering is employed in order to ensure that the most stable form of cocoa butter crystallization occurs. Cocoa butter can crystallize in six different forms, only one of which is desired for the production of quality chocolate. The polymorphous crystallization is controlled by temperature in order to create conditions suitable for only type V crystals to form, which occurs at about 34° C.

The chocolate is first heated to 45° C. (115° F.) to melt all six forms of crystals. It is then cooled to about 27° C. (80° F.), which allow only crystal types IV and V to form. At this temperature, the chocolate is agitated to create many small crystals which will serve as “seeds” to create small crystals in the chocolate. The chocolate is then heated to about 31° C. (88° F.) to eliminate any type IV crystals, leaving just type V.

The final steps would impart efforts to produce a consumer ready product as in any of the various forms and manners of other variants of cocoa butter based confections, or chocolates, and as outlined in the following section.

### Preferred Embodiments

The preferred embodiment is as a stand-alone product; a unique chocolate, as in a traditional candy bar or bon bon, highlighting individual fruits, fruit combinations or fruit/botanical combinations. It may additionally be used in manners consistent with chocolate candy making, which would be familiar to those skilled in the art, such as the enrobing of nuts, caramels, jellies, etc.

Other preferred embodiments would be the product as a component in a product that includes other forms of ‘chocolates’ as described in the U.S. Code of Federal Regulations, Title 21, Chapter 1, Part 163—COCOA PRODUCTS—Subpart B, such as layers of fruit chocolate affixed to a ‘milk chocolate’ layer or a ‘white chocolate’ layer by the interlacing matrix of cocoa butter crystals that form when these layers set from a plastic stage to a semi-plastic stage. Such embodiments shall bear an appropriate statement on its labeling, ie. “With Milk Chocolate”, or “_and White Chocolate”

Still another preferred embodiment would include the product coated in a candy shell, confectioners glaze or wax, to prevent the chocolate from melting in the hands of the consumer. Additionally, cocoa powder may be employed as a coating.

Other preferred embodiments would include the product as a component to another product, such as a baking chip or coating; or sandwiched between biscuit, cookie, wafer, cracker, or pastry layers.

In still other preferred embodiments, inclusions may be admixed to, or otherwise enveloped by, the product, including nuts, ground nuts, dried fruit, freeze-dried fruit, candied fruit, cocoa nibs, wafers, cookies, pretzels, biscuits, crisped/puffed grains, candy pieces (nougat, toffee, hard candy, taffy, candy coated chocolate, etc.) and/or encapsulated liquids prior to letting the product set.

Additionally, a preferred embodiment of the present invention would be as a vehicle to aid in the delivery of compounds to the consumer to address nutrient deficiencies, physical ailments, dysfunctions, maladies and/or diseases; such as ingestible and lipid-soluble essential nutrients, medicaments, formulations, naturally-derived drugs or FDA approved pharmaceuticals, that are intimately incorporated into the product. The inclusion of such additional components are of a percentage consistent with RDA values and/or dosages as set forth by the FDA per serving, if any.

Further, any modifications to the preferred embodiments described herein may be made thereto without departing from the spirit and scope of the present invention.

Throughout this specification and the claims which follow, unless the context requires otherwise, the word “comprise”, and all its variations, will be understood to imply the inclusion of a stated step or group of steps, but not to the exclusion of any other step or group of steps.

1. An intimately incorporated cocoa butter based confection comprising anhydrous fruit based components, as well as nutritive carbohydrate sweeteners, dairy and emulsifiers; whereby the preparation of the intimate incorporation, in the absence of cocoa solids, is a means to highlight a fruit, fruit blend, or fruit/botanical blend in a homogenized melt-away confection while providing healthful benefits.

2. A confection product of claim 1, wherein said fruit based component is defined as being derived from the developed ovary of a seed plant; and
whereby said component comprises:
A. Whole fruit
B. Juiced fruit;
and which may further comprise:
A. Fruit-based acids; whereby said acids comprise those originating in nature, as well as those isolated and/or replicated in a lab.
B. Flavorings
(i) Natural flavors
(a.) Fruit oils
(b.) Fruit essences
(ii) Isolated and replicated nature-identical compounds

3. A confection product of claim 2, wherein said whole fruit and/or juiced fruit component is rendered anhydrous through freeze-drying.

4. A confection product of claim 2, wherein said flavorings are suitable for incorporating into a fat base and the inclusion of which are of predetermined quantities as a means of producing a product with superior palatability and consistency of flavor.

5. A confection product of claim 1, wherein the inclusion of said acids are of predetermined quantities that serve as a means of producing a product with superior palatability and consistency of flavor.

6. A confection product of claim 1, wherein said fruit is selected from the group consisting of the known culinary fruits, including but not limited to: apples, pears, grapes, drupe fruits (ie. peaches, apricots, nectarines, plums, pluots, cherries, greengages, apriums, and peacotums, [etc.]), citrus fruits (ie. mandarin, tangelo, orange, lime, lemon, meyer lemon, clementine, grapefruit, pomelo, blood orange, calamansi, [etc.]), berries (ie. raspberries, strawberries, blueberries, blackberries, loganberries, lingonberries, cranberries, red currants, black currants, acai, gogi, bilberries, boysenberries, huckleberries, salmonberries, acerola, [etc.]), papaya, cactus fruits (pitaya, prickly pear), melons (ie., honeydew, cantaloupe, canary, watermelon, galia, [etc.]), pumpkin, avocado, guava, cherimoya, pomegranate, banana, kiwi fruits, palm fruits, persimmon, tamarind, mangoes, pineapples etc. and mixtures thereof.

7. A confection product of claim 1, wherein the percentage of said product comprised of said fruit component is <40%.

8. A confection product of claim 1, wherein the size of the particle of the intimately included anhydrous fruit component is reduced to <50 μm wide through milling, and <15 μm wide through the further conching process, as a means for producing a product with a homogenous, smooth texture and mouth-feel.

9. A confection product of claim 1, wherein said dairy component is selected from the group consisting of milk, dry whole milk, concentrated, evaporated, condensed or powdered milk/nonfat milk, milkfat, cream, butter, malted milk, buttermilk, dried buttermilk, concentrated buttermilk, whey and/or whey products.

10. A confection product of claim 1, wherein said emulsifying agents, used singly or in combination, do not exceed 2 percent by weight.

11. A confection product of claim 1, wherein said botanical blends are achieved by incorporating anhydrous plant-based culinary additives as a means for providing an additional health benefit, flavor component or natural colorant.

12. A confection product of claim 7, wherein said incorporated anhydrous plant-based culinary additives are selected from the group consisting of: vanilla bean and spice extractives, ground herbs, ground spices, root extractives, powdered stevia extract, green tea powder, black tea powder, bee pollen, powdered soymilk, chlorophyll, freeze dried beet juice, malted cereal extracts, freeze dried wine, coffee, kola, guarana extractives, yerba mate, powdered flowers [etc.] and mixtures thereof.

13. A confection product of claim 1, which further comprises additional anhydrous saccharides; wherein said additional saccharides may be included as an additional sweetener or flavor component selected from the group consisting of: crystallized honey, turbinado sugar, dried agave cactus syrup, maple syrup crystals, dried brown rice syrup, caramelized sugar; and/or may be included as a prebiotic selected from the group consisting of: soluble dietary fibers, inulins, fructo-oliogsaccharides (FOS), polydextrose and galacto-oligosaccharides (GOS), such as lactose, tagatose, [etc.]; and/or as an anti-caking agent, such as maltodextrin, during the freeze drying phase with said juiced fruit.

14. A confection product of claim 1, which further comprises any natural and safe food-grade colorant suitable for incorporating into a fat based product.

15. A confection product of claim 1, and further comprises any essential nutrients, anhydrous medicinal formulations, naturally derived metabolic compounds, or federally approved pharmaceuticals in so long as it retains FDA approval, suitable for incorporating into a fat based product; wherein said confection product acts as a means through which such additive components are solubilized and delivered to the consumer in order to address nutrient deficiencies, physical ailments, dysfunctions, maladies, and/or diseases: and whereby such additives are clearly denoted on labeling.

16. A confection product of claim 14, wherein said essential nutrients are selected from the group consisting of vitamins, minerals, biogenic amines and EFAs.

17. A confection product of claim 14, wherein said metabolic compounds are selected from the group consisting of caffeine, theobromine, theophylline, lycopene, beta-carotene and betulin.

18. An intimately incorporated cocoa butter based confection prepared in the absence of cocoa solids as a means to highlight a fruit, fruit blend, or fruit/botanical blend; whereby said intimate incorporation is arrived at by reducing the particle size of the included anhydrous fruit based components, nutritive carbohydrate sweeteners, dairy components, emulsifiers and any anhydrous botanical components, if at all, to a particle size <15 μm wide through the conching process.

19. A confection product of claim 17, wherein said intimate incorporation may further comprise inclusions that have been admixed to, or are otherwise enveloped by, the product following the conching process.

20. A confection product of claim 18, wherein said inclusions are selected from the group consisting of nuts, ground nuts, dried fruit, freeze-dried fruit, candied fruit, candied ginger, cocoa nibs, wafers, cookies, pretzels, biscuits, crisped/puffed grains, marshmallow, encapsulated liquids and candy pieces, including nougats, toffees, taffys, gasified candies, gummies, jellies, [etc.].

21. A confection product of claim 17, wherein said intimate incorporation may further be affixed to another ‘chocolate’ layer, ie. sweet chocolate, milk chocolate, white chocolate, [etc.] by the interlacing matrix of cocoa butter crystals that form when these layers set from a plastic stage to a semi-plastic stage.

22. A confection product of claim 17, which may further comprise a candy shell, confectioners glaze, wax or cocoa powder coating following the formation of the interlacing matrix of cocoa butter crystals that form when said product sets from a plastic stage to a semi-plastic stage.

23. A method of intimately incorporating into a cocoa butter based confection, in the absence of cocoa solids, a fruit, fruit blend, or fruit/botanical blend; whereby said confection provides healthful benefits; and whereby said intimate incorporation is arrived by reducing the particle size of the included powdered anhydrous fruit based components, nutritive carbohydrate sweeteners, dairy components and other anhydrous botanical components, if at all, to a particle size <15 μm through a conching process with a predetermined and fixed conching pressure as a means of producing a product with a homogenous, smooth texture and mouth-feel.

24. A cocoa butter based confection of claim 23, which may further comprise any emulsifying agent, used singly or in combination, and which does not exceed 2 percent by weight; whereby said emulsifying agents are included as a means of producing a smoother end product, and further as a means to reduce the conching time.
